(19) United States
(12) Patent Application Publication
(10) Pub. No.: US 2005/0019935 A1
(43) Pub. Date: Jan. 27, 2005

(54) Apparatus for staining 2-D electrophoresis gels

(76) Inventors: Martin Andrew O'Toole, Doylestown, PA (US); Artur Matuszewski, Doylestown, PA (US)

Correspondence Address: Martin O'Toole, P.O. Box 93, Doylestown, PA, 18901, US

(21) Appl. No.: 10/626,073

(22) Filed: Jul. 24, 2003

(51) Int. Cl. G01N 33/00

(52) U.S. Cl. 436/63; 422/68.1

(57) ABSTRACT

A system for automatically staining electrophoresis gels by utilizing an active re-circulation system and automatic level control within a modular treatment cell. The gels to be stained are placed within the treatment cell. The control console introduces specific reagents into the treatment cell. The amount of reagents introduced into the treatment cell is controlled by the level control system, which is incorporated into the treatment cell. When the level control system has determined that the cell is full, an active re-circulation is started which ensures that the entire gel surface is exposed to the reagent. On completion of the active re-circulation phase, the treatment cell is automatically drained. Verification that the treatment cell is drained is confirmed by the treatment cell level control system.

### CROSS REFERENCE TO RELATED APPLICATIONS

U.S. Pat. No. 6,398,047 Apparatus for staining electrophoresis gels. Filing date Jul. 23, 2002 U.S. Pat. No. 5,458,749 Device and Process for Staining Electrophoresis Gels, date of patent Oct. 5, 1995

### STATEMENT REGARDING FEDERALLY SPONSORED RESEARCH OR DEVELOPMENT

This invention was made without United States Government support.

### BACKGROUND OF INVENTION

The present invention relates to apparatus and to a method for carrying out the automated staining of electrophoresis gels. Many chemical and biological assays rely upon inducing a change in the optical properties of a protein being tested. Staining of electrophoresis gels is a method for locating the spatial position of proteins, which have been induced to migrate through the gel substrate during electrophoresis. The coordinate position of the protein within the gel is then used to determine relative physical properties of the proteins, such as molecular weight. Many staining materials and techniques have been developed. A few examples are silver staining, and coomasie blue staining.

A significant disadvantage of existing manual staining techniques is that they are open to human error because of their subjective nature. These techniques are also not suited for to uses where a high throughput of samples is required, and are thus relatively expensive to use. The cost factor is exacerbated because, more often than not, different equipment is required for each particular technique.

An object of the present invention is to provide a technique for carrying out staining of electrophoresis gels, which overcomes or at least mitigates certain of these disadvantages. It is also the object of the present invention to provide a system, which allows for high speed automatic staining which is versatile enough to allow it to be used for a variety of different studies.

### BRIEF SUMMARY OF THE INVENTION

The present invention is to an automated apparatus for staining of electrophoresis gels that have already undergone one or two-dimensional electrophoresis. The invention performs automatically, a sequence of events that includes the exposure of the gel slabs to the various reagents and chemicals required, to allow the proteins, which have migrated through the gel slab, and are now located throughout the gel slab, to be visualized. The invention describes a novel gel treatment cell, which relies on fluid flow to insure proper staining.

The invention will expose each of the gel slabs present in the complete treatment cell to each of the chemicals or reagents in turn.

### BREIF DESCRIPTION OF THE SEVERAL VIEWS OF THE DRAWINGS

The following is a brief description of the drawings, in which:

FIG. 1 is a cutaway view showing the fluid path within a fully assembled treatment cell.

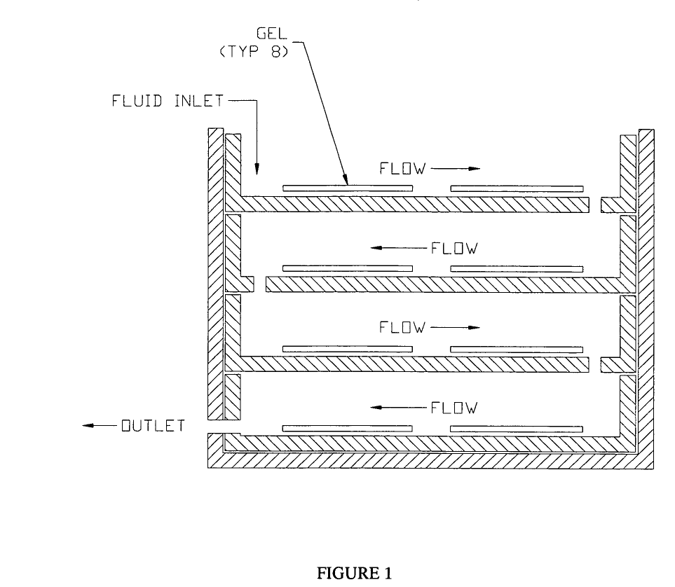

FIG. 2 is an exploded view of a fully assembled treatment cell.

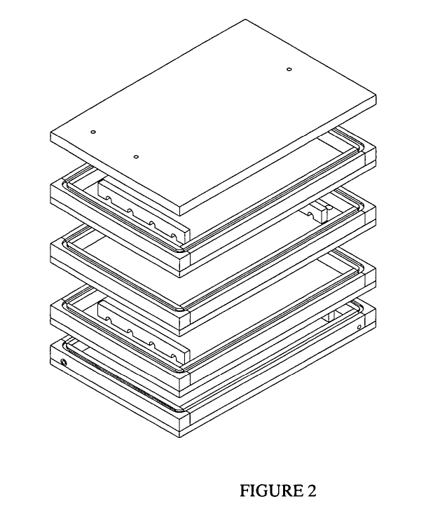

FIG. 3 is a schematic view of the complete system.

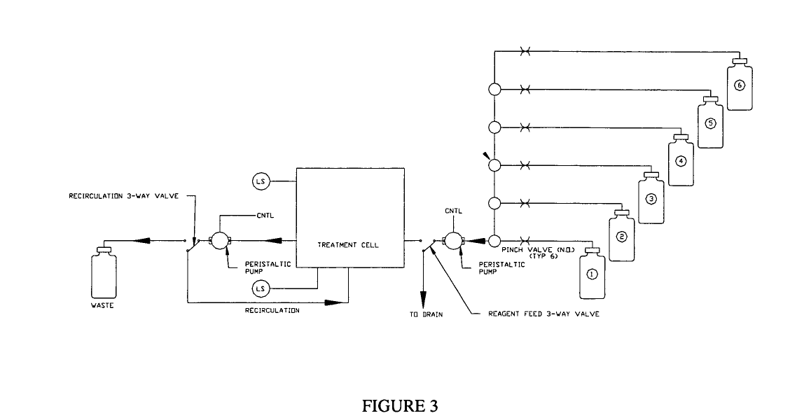

FIG. 4 is a view of the bottom tray.

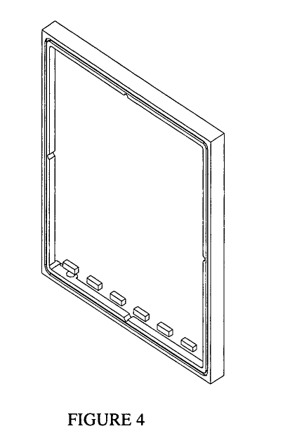

FIG. 5 is a view of the intermediate tray.

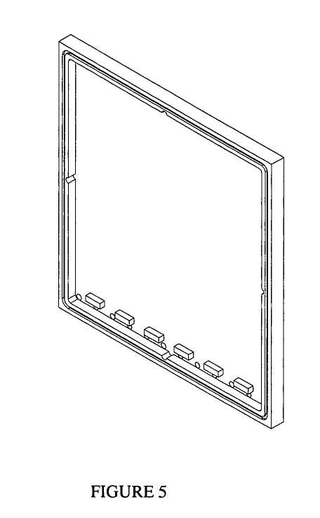

FIG. 6 is a view of the top plate.

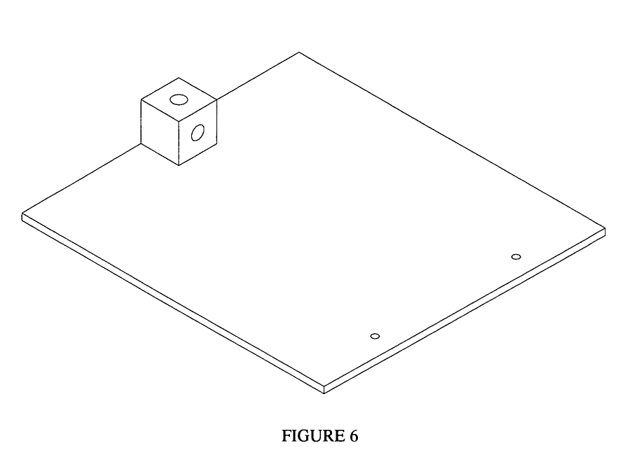

FIG. 7 is a view of the top plate level detector chimney.

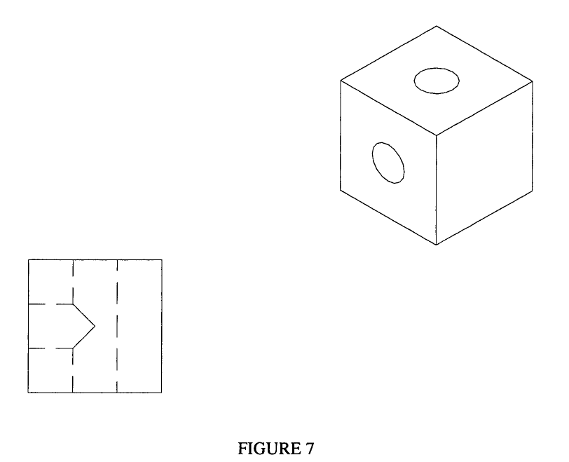

FIG. 8 is a view of an intermediate or bottom plate showing various partitions

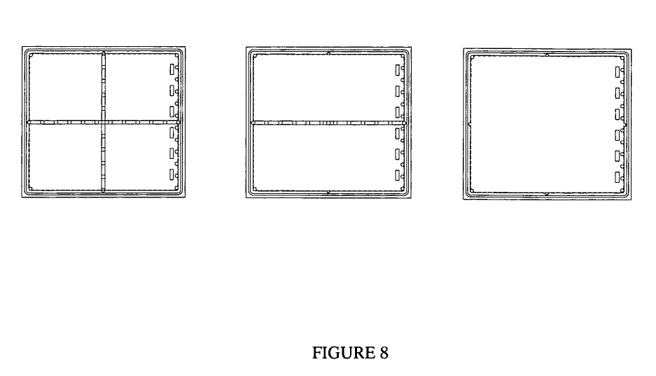

FIG. 9 is a view of stained electrophoresis gels.

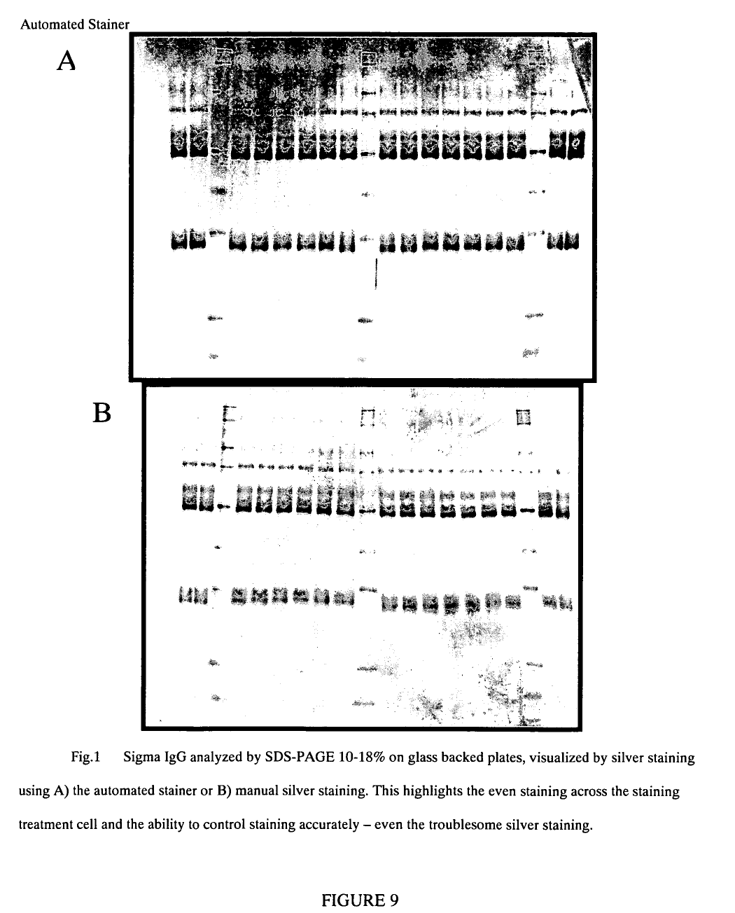

FIG. 10 is a view of stained electrophoresis gels.

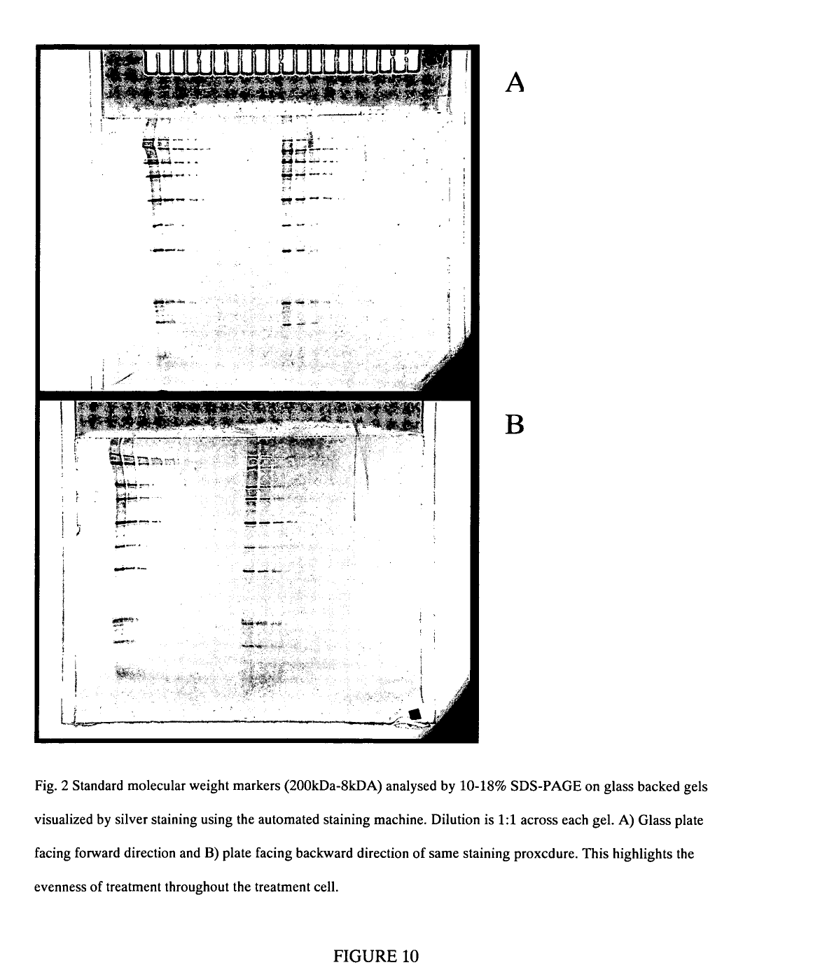

FIG. 11 is a view of stained electrophoresis gels.

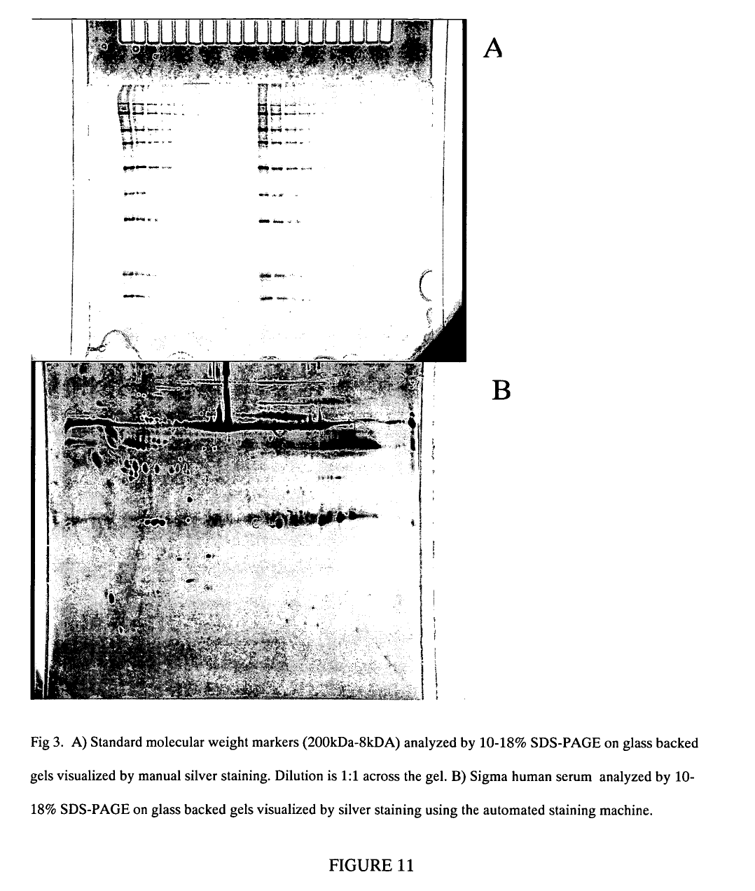

### DETAILED DESCRIPTION OF THE INVENTION

The system is comprised of a control console, which contains all of the microprocessor based control elements, the operator interface, and all of the fluid handling components, with the exception of the treatment cell.

The system also includes a detachable, modular treatment cell.

The treatment cell can take a variety of forms, depending on the format of gels being subjected to the staining method. The treatment cell is comprised of three distinct parts, the top plate, multiple intermediate plates, and the bottom plate. The combination of the three parts results in a single flow through treatment chamber for exposing multiple individual gel slabs to reagents in a staining protocol.

Typically, the top plate resides on the top of the treatment cell and forms a lid for the entire cell. It is also used for the detection of fluid when the cell is filled, and is equipped with both fluid inlet, and air outlet porting. The top plate level detection system includes a fluid flow “chimney” which is a short vertical flow path situated above the top plate. An optical fluid detection device is introduced into the chimney in a horizontal orientation. This arrangement allows foam to exit the treatment cell while allowing the level switch to detect only foam-free fluid.

The bottom plate represents the bottom of the active treatment cell. The bottom plate is equipped with an area for hold one or multiple gel slabs. It is also equipped with a fluid outlet connection, and a level sensor that determines when the entire stack is empty of fluid. The level detector resides in a small well, which facilitates the scavenging of fluid out of the bottom tray to minimize the amount of residual fluid after the treatment cell has been drained. The bottom plate is equipped with a row of individual partitions located directly next to the drain and level sensor well. These partitions keep the gel slabs from covering the level sensor or drain, which would obstruct the flow of fluid from one plate to the next. The bottom plate contains an o-ring, which is placed in a circumferential o-ring groove around the perimeter of each intermediate plate. This o-ring allows the intermediate tray to form a fluid tight seal to the intermediate plate (or top plate) above. The bottom plate may be equipped with dividers which convert the plate from a single gel slab capacity to a double gels slab capacity, and a quadruple gel slab capacity.

The intermediate plate is used between the top plate and the bottom plate. The number of intermediate plates which reside between the top plate and bottom plate can range from zero to many, depending on the quantity of gel slabs be stained in a particular staining run. The intermediate trays are designed to nest together. They are also designed to be placed underneath the bottom plate to act as spacers. In this way, the bottom plate is allowed to migrate up through the treatment cell stack. This feature is used to accommodate the number of gel slabs being stained in a particular gel-staining run. The intermediate plate contains an o-ring, which is placed in a circumferential o-ring groove around the perimeter of each intermediate plate. This o-ring allows the intermediate plate to form a fluid tight seal to the intermediate plate (or top plate) above. The intermediate plate may be equipped with dividers which convert the plate from a single gel slab capacity to a double gels slab capacity, and a quadruple gel slab capacity. Each intermediate plate is equipped with a row of holes along one edge. These holes allow fluid to exit the fluid holding reservoir of the plate and descend to the next intermediate plate, or bottom plate, below. Each intermediate plate is equipped with a row of individual partitions located directly next to the row of holes. These partitions keep the gel slabs from covering the row of holes, which would obstruct the flow of fluid from one plate to the next. Each intermediate plate is equipped with a small drain hole located halfway along the front and back edge of the fluid retaining cavity. These holes serve two purposes. They allow air to escape while the complete treatment cell is being filled, and they allow fluid to drain while the complete treatment cell is being drained to minimize the amount of residual fluid after the treatment cell has been drained.

The combination of the top plate, intermediate plate(s) and the bottom plate form a complete gel staining treatment cell. The cell is designed so as to expose gels slabs, which reside in different elevations within the treatment cell to the same flow conditions. Each intermediate plate is designed to include internal porting which, when combined with other intermediate plates, the top plate, and the bottom plate, forms a treatment cell which exposes each level within the cell to the reagent flow in a serial manner. In this way, flow characteristics such as flow rate, cross sectional velocity, and pressure are the same in every tray within a complete treatment cell, regardless of the number of intermediate plates within the complete treatment cell.

In comparison to other staining methods which rely on externally induced physical motion of the treatment cell, or staining tray, to induce a oscillation of the fluid within the treatment cell to properly expose the gel slab to the reagent or chemical, the present invention relies on the cross sectional velocity of the fluid flowing across, over, and under each gel to achieve the same result.

Regardless of the form of the treatment cell, it has the following common characteristics. Each treatment cell consists of a fluid holding chamber that can receive, and retain, reagents, which are pumped from the control console.

The treatment cell also contains an integrated level control system for regulating the introduction and expulsion of reagent from the treatment cell.

The treatment cell also contains the necessary fluidic connections to provide a means of interconnecting the control console and the treatment cell.

The combination of the treatment cell and the control console are used to perform the staining procedure.

The method used by the apparatus is repeated using different reagents to carry out a user defined staining protocol. The basic method is as follows:
1. The desired reagent is introduced into the treatment cell via one of the fluid pumps located in the control console.
2. When the treatment cell level control system determines that the fluid level is sufficient, the fill pump is stopped, and the re-circulation pump is started. The re-circulation pump draws reagent from the bottom of the cell, and it is reintroduced into another portion of the cell to create a general turbulence, which exposed the complete surface of the gel to the reagent. This re-circulation is continued for a length of time determined by the operator of the apparatus.
3. When the treatment cell re-circulation duration timer has expired, the cell is then drained of reagent. The treatment cell level control system determines when the treatment cell is empty, and the next reagent can be introduced.

Steps one through three are then repeated as required, with the reagents required, to complete an entire protocol.

1. An apparatus for staining a plurality of gel slabs, said apparatus comprising: a fluid handling pump for introducing a fluid from a series of fluid reservoirs; a valve manifold which selects which reservoir the fluid handling pump will draw from; a re-circulation pump which actively re-circulates the fluid which has already been introduced by means of the fluid handling pump; a computer control system which executes a staining protocol based on the operator input data; a level control system which allows the system to proceed from one to the next sequence step; a gel slab treatment cell which holds the gel slabs during the staining process; and a clamp mechanism that compresses the treatment cell stack to form a single fluid tight container.

2. The apparatus of claim 1 wherein said gel slab treatment cell consists of three distinct modular parts, a top plate, intermediate plates, and a bottom plate.

3. The apparatus of claim 2 wherein said top plate is equipped with a liquid level detecting sensor in a “chimney”.

4. The apparatus of claim 2 wherein said top plate is equipped with a fluid inlet manifold.

5. The apparatus of claim 2 wherein said top plate is equipped with an air outlet vent.

6. The apparatus of claim 2 wherein said intermediate plate is equipped with a fluid holding reservoir.

7. The apparatus of claim 6 wherein said fluid holding reservoir is equipped with a row of partition blocks.

8. The apparatus of claim 2 wherein said intermediate plate is equipped with a shoulder on the bottom to allow it to nest into other intermediate plates or bottom plates.

9. The apparatus of claim 2 wherein said intermediate plate is equipped with an o-ring in a circumferential o-ring groove.

10. The apparatus of claim 2 wherein said intermediate plate is equipped with a row of fluid exit ports.

11. The apparatus of claim 2 wherein said intermediate plate is equipped with a drain hole located in the middle of the front and back edges of the fluid holding reservoir.

12. The apparatus of claim 2 wherein said intermediate plate may be equipped with a variety of gel slab capacity changing partitions.

13. The apparatus of claim 2 wherein said intermediate plate is equipped with a row of individual partition blocks.

14. The apparatus of claim 2 wherein said bottom plate is equipped with a fluid holding reservoir.

15. The apparatus of claim 2 wherein said bottom plate is equipped with a fluid outlet connection.

16. The apparatus of claim 2 wherein said bottom plate is equipped with a fluid holding reservoir.

17. The apparatus of claim 2 wherein said bottom plate is equipped with a liquid level sensor.

18. The apparatus of claim 17 wherein said liquid level sensor is equipped with a fluid drain sump.

19. The apparatus of claim 2 wherein said bottom plate is equipped with an o-ring in a circumferential o-ring groove.

20. The apparatus of claim 2 wherein said bottom plate may be equipped with a variety of gel slab capacity changing partitions.

21. The apparatus of claim 2 wherein said bottom plate is equipped with a row of individual partition blocks.

22. The apparatus of claim 1 wherein said valve manifold selects which reservoir the fluid handling pump will draw fluid from.

23. The apparatus of claim 1 wherein said re-circulation pump draws fluid out of the bottom, and then reintroduces said fluid to the top of, the complete treatment cell.

24. A computer assisted method for transferring a chemical or reagent to and from the treatment chamber, wherein said fluid produces a level signal indicating the presence or absence of fluid in treatment chamber and transmitting said signal to a computer.

25. The method of claim 24 further comprising the steps of: filling the chamber with a chemical reagent by means of a fluid-handling pump.

26. The method of claim 25 wherein the filling is performed until the level switch in the top plate indicates that the complete treatment cell is filled.

27. The method of claim 24 further comprising the steps of: re-circulating the fluid contained within the treatment chamber with a chemical reagent by means of a fluid-handling pump.

28. The method of claim 27 wherein the re-circulation is performed until a timer elapses in the computer.

29. The method of claim 24 further comprising the steps of draining the chamber of a chemical reagent by means of a fluid-handling pump.

30. The method of claim 24 further comprising the steps of draining the chamber of a chemical reagent by means of a fluid-handling pump.

31. The method of claim 30 wherein the draining is performed until the level switch in the bottom plate indicates that the complete treatment cell is drained.
